SEQUENCE LISTING (13 sequences)

SEQ ID NO: 1 - DNA, 4785 bp, Homo sapiens
  CDS (1875)..(2954)
acctgcttccctctcggatagccagggctgatgatttcaactcattaatcattccgaactcttcccaacaaacagcctcatggctgtcccggacctcctctgtcccggctcaggaaatgatctcggtcggaagtgaaaagaccaaatcggccccaggaggccattttcttcgacagaagctgaggcgtgggggcgtggccttcccccgcttccccctccactcccccgcccgcccccggccccggctttccgatctcggcccagggaagagccttggtcgcgaatcaccagtttgcatttgcccctggggcgggggcggctttctgagaccaggctggggctgggactccgccaccttcagctaagggtaccgaggtcagtccgggagtcgcgtcaccgggactcgaacccgcctcttcgtcttccgaggcctagtgcgagcctgcggcgctaaacgcagtcacactgtccctttaaggcggcactttttattcttcatttgtttacttcttcatgcgctcttcggtctttacaaaatgtgctcgcgatatccttccgcaagtgtcagtctcaggcactcaccacccgccttgaagccatttctataatcttaagactcgcgagttagcagccgtgggccgacgtctctctttataggatttaattcttttacattttagggactactttttaggaataaaccattcttgcgttagttaataaaggttcataaccttcggaacccctccgctgagaacaaaatacgataatagcctttataacgcaccttttcataaaaatattctgtgaggactctcataggccatcgaatcttctcgtattcctgaaacagaatggtacggtccagaacccgccccatcttctctgcgattggccgtccttcctcagcgagtcgtgtgatttttcaccaatggccgctcgcgtttctttagcgaggtctaagcgatggaaggtggcggccagggagacgccccctacgcgtcctgggattggctacgtcgcacggcgcgcgagggcggcgggcccgaaagataagaactacgactcccggcgccccgcaaagggaactccgttacgcatgcgcacgggcggggcgaaaaagcttctatataaaaggggcgcagaggactgggagacagcagttggaagttggcaggtggagaggcaggttgggagggaaagtcgggggaggacgcggaagaggagctgtgggaagggggaggagggagggaggaaaagaggaggaggcggaggagaactgagcagagcagagcatcgagccaaaggggagatgagtttgtctgtcctctgctgaggctacggccgggcctagggaactgggagcttgggtggaagcgacacccgtggaagtgggaggaggtggcgccgggactttaaccccttgtgggctctgcggcaggggatttaaccctttgtggatctggcccctcggaggcagcgtcatcggtagttttaaccccttcggggctgggtttcacgcactggacttaccctcatcaccttgctcaccaactcctttattggggtgctccgcttggaggtttgaggcccacctccgcccattacgtactgttcctgccgctgcacccccttggacccgctagctggccgcactgtgggcgcttaaccctttactgacttgagctccccagattgcagttggagtttgctgatagaaggactagctaaaggcgtcactgcaggaattacaaactgaagaggactctgttggactgttttttttttctttttcttttttttaagaaaaacccatttttttccttaaggacttactagccaaaatttcttaaacttcgaggactctactagccatggccgagccattcttgtcagaatatcaacaccagcctcaaactagcaactgtacaggtgctgctgctgtccaggaagagctgaaccctgagcgccccccaggcgcggaggagcgggtgcccgaggaggacagtaggtggcaatcgagagcgttcccccagttgggtggccgtccggggccggagggggaagggagcctggaatcccaaccacctcccttgcagacccaggcctgtccagaatctagctgcctgagagagggcgagaagggccagaatggggacgactcgtccgctggcggcgacttcccgccgccggcagaagtggaaccgacgcccgaggccgagctgctcgcccagccttgtcatgactccgaggccagtaagttgggggctcctgccgcagggggcgaagaggagtggggacagcagcagagacagctggggaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcaaaaccggcctgtactccaagcgggccgccgccaaatccgacgacaccagcgatgacgacttcatggaagaagggggtgaggaggatgggggcagcgatgggatgggaggggacggcagcgagtttctgcagcgggacttctcggagacgtacgagcggtaccacacggagagcctgcagaacatgagcaagcaggagctcatcaaggagtacctggaactggagaagtgcctctcgcgcatggaggacgagaacaaccggctgcggctggagagcaagcggctgggtggcgacgacgcgcgtgtgcgggagctggagctggagctggaccggctgcgcgccgagaacctccagctgctgaccgagaacgaactgcaccggcagcaggagcgagcgccgctttccaagtttggagactagactgaaacttttttgggggagggggcaaaggggactttttacagtgatggaatgtaacattatatacatgtgtatataagacagtggacctttttatgacacataatcagaagagaaatccccctggctttggtttcgtaaatttagctatatgtagcttgcgtgctttctcctgttcttttaattatgtgaaactgaagagttgcttttcttgttttcctttttagaagtttttttccttaatgtgaaagtaatttgaccaagttataatgcatttttgtttttaacaaatcccctccttaaacggagctataaggtggccaaatctgagaacaattaaattcattttagttataataaatttaatatttgtaaatgtaacatagtttcagtgtgatttctagagctaattcaaaatagtattgatatattttatgtgactgcatttttggggaggggtaccgaaatcgttaaatttgtcagtttgcaaaaatatcaatctttaatgggagaattttcaatttgccaattttttccttgaatgggtttaagtatgctacaatatacagttcaggcaaaatttaagatgtaattatcttcaatacttaagtgtgcttgctttctagtgccttggttttctttcttgatgctggaaaaataaacaaaccggtattgagtgtttaggcgagtggaaagtggctacaatccaaaattttaaatttaactctgcctcggccattcaaaagtctaataacaaaaaatgtaaacctaatttggcagtttgttaggttagacaactgacagcctcatttcattcctacaagttggttttcagtaatctcttccttccccccagtaaggctggaagaggctcttggcaaacttcttagtgcaagcaatggttagattaatttgtgaggcagctctttaagacgttcagaggtaagaaatactggatttataaagcaaatggctgtttgggggattccaaggatttacctaattgtccaattctacgtgctctctataccaaaacaaaaaaaaaaagctatccacctttccatgtgggtcaaactaaaattagaaatgtcccctcactgcagatcaaatgtaaagcttccagttaaggagctaaatgaggtcctcagctgaatgaggaaccctgtacatccccttgcacagccctattctaaatcgcttaaactatgctgatagctgcttaggttcttgagtagttctgctcttaaacgtagggaggccctgagaactaaattttgccccaaaataaaaacagaaattatgagattgcctcctgtcattttggttaacccagtccttcacctgccctgtgtcagtgtcttctgagggcaattgcgttgctcaaatcactagcacagaggttccttaatttggggccttagaaaccattgtgggccttggggtccatgaaccccatgaaattatttgtagacttgtatgtacatttttctggggagaaggttcaagagattcataagattgtcaaactccttgaaggttcagaacctctgcagggaagggggaagaaaaccctcccattaggaagcatgcttttgcagttaaatggcgatggtggaggtgatagggacttcaagagtaaaatgcaccttgtattgcataagaagcatacacaaatcaataaatcaagggagattataccagtaggactgaatcagggccttcaaagctggactgagttggtcctgttctggcacatatggtccactggagacaatgtatgattgagtttttctttggtctaaaaattatattaaacatttattttgaaata

SEQ ID NO: 2 - protein, 359 aa, Homo sapiens
MAEPFLSEYQHQPQTSNCTGAAAVQEELNPERPPGAEERVPEEDSRWQSRAFPQLGGRPGPEGEGSLESQPPPLQTQACPESSCLREGEKGQNGDDSSAGGDFPPPAEVEPTPEAELLAQPCHDSEASKLGAPAAGGEEEWGQQQRQLGKKKHRRRPSKKKRHWKPYYKLTWEEKKKFDEKQSLRASRIRAEMFAKGQPVAPYNTTQFLMDDHDQEEPDLKTGLYSKRAAAKSDDTSDDDFMEEGGEEDGGSDGMGGDGSEFLQRDFSETYERYHTESLQNMSKQELIKEYLELEKCLSRMEDENNRLRLESKRLGGDDARVRELELELDRLRAENLQLLTENELHRQQERAPLSKFGD

SEQ ID NO: 3 - DNA, 3401 bp, Mus musculus
  CDS (598)..(1668)
gaggaaaagaggaggaggcggaggagaactgagcggagcatcgagccagaggggagatgagtttgtctgtcctcggctaaggctccggcccggcctgaggaactgggagctcgggttggagcgacacccgtggaagtgggaggtggcggctctgggactttaaccccttgtgggctctgcggcaggggatttaaccctttgtggattcggcccctccgaggcagcgtcatcgggtagttttaaccctttcgggttgggtttaacccattggacttaaacctcctctccttgcgcaccaactcctcgtctgtgggagcactcggcggggaggcgtgaagcccacccctctgcatatttgcatacggtgcctgctgctgaacccccggacctgctagttggccacgcttgtgtgcgcttaaccctttattgactggactccaagttgcgattggagtttgctgacaggacggacggaaggcgtcactgcagaaattactgaccgaagaggactttgttggacatattttgaaaagaaagaaaaacctctttccttaagccagaatttctcaaacttccagaagagaactctactaagccatggccgagccactcttgacagaacatcaacaccagcctcaaactagcaactgtacaggtgctgctgttgtccatgaagagcatacctctgagcgccccccaagcgcggaggaacgggtgcccaaggaggacagtaggtggcaatcgagagcgtccttgcagtccggtagccgtccagggcaggagggagaagggggcctgaagcaccagctgcccccattgcagacgaatgcctgtccagaattgagctccctggagaagggcgagaagggccagaatggggaggacttatccactggcggtgcctccccgtcggcggagggagagccgatgtcagagtccctcgtgcagccaggtcatgactcggaggccaccaagcaggaggctcctgccgccggaggcgaggagccatggggacagcaacagagacagctgggcaagaaaaaacatcggagacgcccctcaaagaagaagcggcattggaagccctactacaagctgacttgggaggagaagaaaaagttcgacgagaagcagagcctgcgagcttcgcgggttcgagccgagatgttcgccaagggccagccagttgcgccctataacaccacgcagttcctcatggatgaccacgatcaggaggagcctgatctcaaaaccggcctttaccccaagcgggcagccgccaaatccgacgacaccagcgatgaggattttgtggaagaagctggtgaggaagacggaggcagcgatggcatgggaggggacggcagcgagtttctgcagcgggacttctcggagacgtacgagcggtaccacgccgagagcctgcagaacatgagcaagcaggagctcatcaaagagtacctggagctggagaagtgcctctcccgcaaggaggacgaaaataaccggctgcggctggaaagcaagcggctgggaggcgtcgacgcgcgagtgcgggagctcgagctagagctggaccggctgcgcgctgagaacctccagctgctgaccgagaacgaactgcaccggcagcaggagcgagcgcctctttccaagttcggcgactagactgaaaacttttggggggagggggcaaaggggactttttacagtgctggaatgtaacattatatacatgtgtatataagacagcggacctttttatgacacataatcagaagagaaaatcgccccggctttggtttggtaaatttagctatgatgttgcttgtgtgctttctcctgttctttaattatgtgaaactgaagggttgcttttcatgttttcctccttagatgttgtttcttaatgtgcaagtaatctgaccaagttataatgcatttttctgttttaaaaaaaaaatcccctccttaagcggagctataaggtggccaaatctgagacccattacattcattctagttataataaatttaatgtttgtaaatgtaacagtttcagtgtgatttctagagattaaaaatcgtgctggtttgtgtgactgcacttttgaggtggggtaccaagattgttaaatttatctatttgcaaaaatacttgaatagaagaaattcagtttgttccctccacccccaccaatgggtttttaaattttcaaaatacagttcaggtaaaatgtactcaatgttgtgcctgcttcctaatgctggggagtagaaagtggctacaggcagcagcttagcttgcgcaccttgatggctgttcaaaagagtgctaacaaaagtataagcctgatttggcaattgttagataacgaaaagcctcatttcatctccacaagtcaatttttcagtaatcctggcttccgtcctaaccaccccacaaaattccaatggagttcctgtttgcctaccttggtaagtaaggctcctggtaaacttgctttcttagtacatccaagagatagccaagataaacttgtgagaaaacatgttagcgatatgttggacttcaaatggctaagtgatctaaggacttagcatcaatgtctgacacttaagtactttttctattgaaaataagaaagaaaagctgccctttccattagaatccaactgaaatttgaaatgcccctttctacaagtaaagctggctcaggagcgggctgaggtctttagatgatgaatacaaaccctgcatgtttccagtactgcagctattctcaatctcctgggctgcctggacagctgcctaggttaggctgctcttgaaaatgcagggaggctctgagaactaggccttatcacagaatcaaaatggtaaactcaacagttctctcatgtcagtatagttaatgcaaccctgtcacctgacctgtgtcagtgtttccttagggcaattatattgctaaaattattagcacaactggctccgtaacttggagtcttaaaaaccatagggggccttgggggtccacaagccccacataattgtgtgaatatatatatatataatatatatatatatatattgaatatattatatatattcaagtgggttcaagaactcaggaggttgtcaaattccttggtggttaaaaaccccttagggcaggggcttctcagccttcctgccagctaaatggtaacagtggagttgactggaacatcaagaacgaatgcagctacaaatcaataatcaagaaaagctgtgccaggaggacggcttgggaccttcaaaaccaagcttgctggttctattctggcaggtggtctcctgcagaaactgtaaaatcaacttttctttgatctaaagattgcattaaatgtctattttgaaa

SEQ ID NO: 4 - protein, 356 aa, Mus musculus
MAEPLLTEHQHQPQTSNCTGAAVVHEEHTSERPPSAEERVPKEDSRWQSRASLQSGSRPGQEGEGGLKHQLPPLQTNACPELSSLEKGEKGQNGEDLSTGGASPSAEGEPMSESLVQPGHDSEATKQEAPAAGGEEPWGQQQRQLGKKKHRRRPSKKKRHWKPYYKLTWEEKKKFDEKQSLRASRVRAEMFAKGQPVAPYNTTQFLMDDHDQEEPDLKTGLYPKRAAAKSDDTSDEDFVEEAGEEDGGSDGMGGDGSEFLQRDFSETYERYHAESLQNMSKQELIKEYLELEKCLSRKEDENNRLRLESKRLGGVDARVRELELELDRLRAENLQLLTENELHRQQERAPLSKFGD

SEQ ID NO: 5 - protein, 7 aa, Artificial Sequence
  RNA Pol II consensus sequence
YSPTSPS

SEQ ID NO: 6 - DNA, 643 bp, Homo sapiens
gcccactgatgaggaagggcctctgcgggtgagacctgcttccctctcggatagccagggctgatgatttcaactcattaatcattccgaactcttcccaacaaacagcctcatggctgtcccggacctcctctgtcccggctcaggaaatgatctcggtcggaagtgaaaagaccaaatcggccccaggaggccattttcttcgacagaagctgaggcgtgggggcgtggccttcccccgcttccccctccactcccccgcccgcccccggccccggctttccgatctcggcccagggaagagccttggtcgcgaatcaccagtttgcatttgcccctggggcgggggcggctttctgagaccaggctggggctgggactccgccaccttcagctaagggtaccgaggtcagtccgggagtcgcgtcaccgggactcgaacccgcctcttcgtcttccgaggcctagtgcgagcctgcggcgctaaacgcagtcacactgtccctttaaggcggcactttttattcttcatttgtttacttcttcatgcgctcttcggtctttacaaaatgtgctcgcgatatccttccgcaagtgtcagtctcaggcactcaccacccgccttgaagccatttctataatc

SEQ ID NO: 7 - DNA, 798 bp, Homo sapiens
aacgcaccttttcataaaaatattctgtgaggactctcataggccatcgaatcttctcgtattcctgaaacagaatggtacggtccagaacccgccccatcttctctgcgattggccgtccttcctcagcgagtcgtgtgatttttcaccaatggccgctcgcgtttctttagcgaggtctaagcgatggaaggtggcggccagggagacgccccctacgcgtcctgggattggctacgtcgcacggcgcgcgagggcggcgggcccgaaagataagaactacgactcccggcgccccgcaaagggaactccgttacgcatgcgcacgggcggggcgaaaaagcttctatataaaaggggcgcagaggactgggagacagcagttggaagttggcaggtggagaggcaggttgggagggaaagtcgggggaggacgcggaagaggagctgtgggaagggggaggagggagggaggaaaagaggaggaggcggaggagaactgagcagagcagagcatcgagccaaaggggagatgagtttgtctgtcctctgctgaggctacggccgggcctagggaactgggagcttgggtggaagcgacacccgtggaagtgggaggaggtggcgccgggactttaaccccttgtgggctctgcggcaggggatttaaccctttgtggatctggcccctcggaggcagcgtcatcggtagttttaaccccttcggggctgggtttcacgcactggacttaccctcatcaccttgctcaccaactcctttattggggtgctccgctt

SEQ ID NO: 8 - DNA, 1207 bp, Homo sapiens
aaacttcgaggactctactagccatggccgagccattcttgtcagaatatcaacaccagcctcaaactagcaactgtacaggtgctgctgctgtccaggaagagctgaaccctgagcgccccccaggcgcggaggagcgggtgcccgaggaggacagtaggtggcaatcgagagcgttcccccagttgggtggccgtccggggccggagggggaagggagcctggaatcccaaccacctcccttgcagacccaggcctgtccagaatctagctgcctgagagagggcgagaagggccagaatggggacgactcgtccgctggcggcgacttcccgccgccggcagaagtggaaccgacgcccgaggccgagctgctcgcccagccttgtcatgactccgaggccagtaagttgggggctcctgccgcagggggcgaagaggagtggggacagcagcagagacagctggggaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcaaaaccggcctgtactccaagcgggccgccgccaaatccgacgacaccagcgatgacgacttcatggaagaagggggtgaggaggatgggggcagcgatgggatgggaggggacggcagcgagtttctgcagcgggacttctcggagacgtacgagcggtaccacacggagagcctgcagaacatgagcaagcaggagctcatcaaggagtacctggaactggagaagtgcctctcgcgcatggaggacgagaacaaccggctgcggctggagagcaagcggctgggtggcgacgacgcgcgtgtgcgggagctggagctggagctggaccggctgcgcgccgagaacctccagctgctgaccgagaacgaactgcaccggcagcaggagcgagcgccgctttccaagtttggagactagactgaaacttttttgggggagggggcaaaggggactttttacagtgatggaatgtaacattatatacatgtgtatataagacagtggacctttttatgacacat

SEQ ID NO: 9 - DNA, 21 bp, Artificial Sequence
  Hexim-1 mRNA silencer sequence
uuccaugaagucgucaucgct

SEQ ID NO: 10 - DNA, 1080 bp, Homo sapiens
atggccgagccattcttgtcagaatatcaacaccagcctcaaactagcaactgtacaggtgctgctgctgtccaggaagagctgaaccctgagcgccccccaggcgcggaggagcgggtgcccgaggaggacagtaggtggcaatcgagagcgttcccccagttgggtggccgtccggggccggagggggaagggagcctggaatcccaaccacctcccttgcagacccaggcctgtccagaatctagctgcctgagagagggcgagaagggccagaatggggacgactcgtccgctggcggcgacttcccgccgccggcagaagtggaaccgacgcccgaggccgagctgctcgcccagccttgtcatgactccgaggccagtaagttgggggctcctgccgcagggggcgaagaggagtggggacagcagcagagacagctggggaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcaaaaccggcctgtactccaagcgggccgccgccaaatccgacgacaccagcgatgacgacttcatggaagaagggggtgaggaggatgggggcagcgatgggatgggaggggacggcagcgagtttctgcagcgggacttctcggagacgtacgagcggtaccacacggagagcctgcagaacatgagcaagcaggagctcatcaaggagtacctggaactggagaagtgcctctcgcgcatggaggacgagaacaaccggctgcggctggagagcaagcggctgggtggcgacgacgcgcgtgtgcgggagctggagctggagctggaccggctgcgcgccgagaacctccagctgctgaccgagaacgaactgcaccggcagcaggagcgagcgccgctttccaagtttggagactag

SEQ ID NO: 11 - RNA, 25 nt, Artificial Sequence
  Hexim-1 miRNA (miR1)
cuguacaguugcuaguuugaggcug

SEQ ID NO: 12 - RNA, 21 nt, Artificial Sequence
  Hexm-1 miRNA (miR2)
augaggaacugcgugguguua

SEQ ID NO: 13 - RNA, 19 nt, Artificial Sequence
  Hexim-1 miRNA (miR3)
uuccaugaagucgucaucg

(19) United States
(12) Patent Application Publication
(10) Pub. No.: US 2015/0152421 A1
(43) Pub. Date: Jun. 4, 2015

(54) HEXIM-1 AS A TARGET OF LEPTIN SIGNALING TO REGULATE OBESITY AND DIABETES

(71) Applicant: MEDHEXIM, LLC, EAST NORTHPORT, NY (US)

(72) Inventors: Eduardo J. Mascareno, East Northport, NY (US); Manya Dhar-Mascareno, East Northport, NY (US)

(73) Assignee: MEDHEXIM, LLC, EAST NORTHPORT, NY (US)

(21) Appl. No.: 14/407,748

(22) PCT Filed: Jun. 14, 2013
(86) PCT No.: PCT/US13/45985

§ 371 Date: Dec. 12, 2014

Related U.S. Application Data
(60) Provisional application No. 61/659,882, filed on Jun. 14, 2012.

(51) Int. Cl. C12N 15/113; G01N 33/50

(52) U.S. Cl. CPC C12N 15/1136; G01N 33/5023; G01N 33/5041; C12N 2310/14; C12N 2310/141; G01N 2500/10

(57) ABSTRACT

Provided are Hexim-1 inhibitor compositions, and methods and kits using said compositions for the treating and regulating obesity and/or diabetes, comprising administering an effective amount of a Hexim-1 inhibitor. Additionally provided are methods for screening for inhibitors of Hexim-1.

### CROSS-REFERENCE TO RELATED APPLICATIONS

This application claims the benefit of priority to U.S. Provisional Patent Application 61/659,882, filed 14 Jun. 2012, which is incorporated by reference herein in its entirety.

### GOVERNMENT RIGHTS

The invention was made with government support under Grant No. HL073399 awarded by the National Institutes of Health. The government has certain rights in the invention.

### FIELD OF INVENTION

Applicants' data demonstrates the role of Hexim-1 in regulating obesity and diabetes. Accordingly, the invention relates to compositions, methods and kits for treating and regulating obesity and diabetes by administering an effective amount of a Hexim-1 inhibitor. The invention also relates to methods for screening for inhibitors of Hexim-1.

### SEQUENCE LISTING

A Sequence Listing (in .txt format) comprising SEQ ID NOS:1-13 was filed as part of this application, and is incorporated by reference herein in its entirety.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The estimated US healthcare costs for obesity are $100 bn to $150 bn annually, with other leading nations following those trends. The incidence and prevalence of diabetes mellitus are on the rise and are closely linked with that of obesity. The World Health Organization estimates the number of diabetics to exceed 350 million by 2030 with an estimated cost of $218 bn a year in the US alone. Taken together, there is an urgent need to identify new target(s) and to develop new therapies against diabetes and obesity.

Leptin is an established link between obesity and diabetes. Leptin is secreted by the fat cells, along with other tissues, to act on the hypothalamic leptin receptors (Ob-Rb) to decrease food intake and increase energy expenditure in the host. Under physiological conditions, the amount of leptin produced by fat tissues is directly related to the mass of adipose tissues. However, leptin deficiency and leptin resistance (more often in a clinical setting) lead to hyperphagia and decreased energy expenditure, characterized by obesity, and type II diabetes (insulin resistance) (1). Leptin directly affects insulin sensitivity by regulating the efficiency of insulin-mediated glucose metabolism by the skeletal muscle.

The binding of leptin to the extracellular domain of the functional leptin receptor isoform (LRb), mediates the activation of the intracellular, LRb-associated Jak2 tyrosine kinase, resulting in Jak2 autophosphorylation on tyrosine residues (pY) as well as the phosphorylation of three tyrosine residues on the intracellular tail of LRb (2). These post-translational modifications induce the recruitment of signal transducer and activator of transcription (STAT) 3, which is activated to mediate transcriptional events, including the transcription of pro-opiomelanocortin (POMC) and the inhibitory suppressor of cytokine signaling 3 (SOCS3) proteins. The activation of the tyrosine kinase Jak2 induces the tyrosine phosphorylation of the Insulin Receptor Substrate-1 (IRS-I) a key downstream component of the insulin signaling, stimulating the insulin action or glucose uptake (3). The transcriptional induction of SOCS3 confers a negative feedback loop: SOCS3 binds to LRb and Jak2 kinase to impair LRb signaling. Genetic models that interfere with expression of SOCS3 are resistant to development of obesity and diabetes (4). On the other hand, increased levels of SOCS3 are a key feature of obesity and diabetes. Taken together, data suggest that leptin, Jak2 kinase and the IRS-I may be the main components that share the responsibility of maintaining a balanced metabolism. In addition to critical function, they also share conserved structural domains, such as the SH2 domain, a specialized phosphotyrosine-binding domain. They associate with adaptor proteins through the conserved SH2 domain, among them the Src homology 2 (SH2) B adaptor protein 1 (SH2B1) (5). Indeed, SH2B1 binds simultaneously to both JAK2 and IRS1 or IRS2, mediating the formation of a complex of JAK2, SH2B1, and IRS1 or IRS2, resulting in the activation of the Insulin dependent PI3-kinase pathway. The primary JAK2-binding site for the SH2 domain of SH2B1 has been identified as phosphotyrosine 813 (pTyr813) which resides in a YXXL motif in a region adjacent to the kinase domain (5). Mutations within this conserved motif disrupt Jak2 kinase autophosphorylation and kinase activity, suggesting that the conserved YXXL motif plays a significant role in regulating obesity and diabetes.

The activation of gene transcription involving RNA polymerase II is characterized by at least three stages, namely, initiation, elongation and termination. Each stage is highly regulated and requires the participation of multiple factors. Serine phosphorylation of carboxyl terminal domain (CTD) of RNA polymerase II (Pol II) is essential in mediating RNA chain elongation in transcription of eukaryotic genes (6). In order for RNA Pol II to enter into the elongation phase, the serine kinase activity of the cyclin dependent kinase 9 (Cdk9) contained within the pTEFb complex is required to phosphorylate the serine residues of the conserved carboxyl terminus domain (CTD) of RNA Pol II with the consensus Tyr-Ser-Pro-Thr-Ser-Pro-Ser (SEQ ID NO:5). Hexim-1 (hexamethylene bisacetamide inducible protein 1) was initially identified as an inhibitor of smooth muscle proliferation and the positive elongation pTEFb complex by interacting with Cyclin Ti, the partner of Cdk9. Only upon release of Hexim-1 (Hexim1) from the p-TEFb (7) complex will Cdk9 phosphorylate the CTD of Pol II and allow transcription elongation to occur.

Hexim-1 gene is highly homologous between human and mouse (accession no. NM_{—}0066460, version no. NM_{—}006460.2 GI:70167225 for the human gene/cDNA/mRNA (SEQ ID NO:1); and accession no. NP_{—}006451, version no. NP_{—}006451.1 GI:5453682 for the human protein) (SEQ ID NO:2) (accession no. NC_{—}000077, version no. NC_{—}000077.6 GI:372099099 for the mouse gene/cDNA/mRNA (SEQ ID NO:3), and accession no. NP_{—}620092, version no. NP_{—}620092.1 GI:20270289, for the mouse protein) (SEQ ID NO:4), and is characterized by a single exon with conserved regulatory regions. Expression of this gene is induced by hexamethylene-bis-acetamide in vascular smooth muscle cells. The human HEXIM-1 gene has no introns and comprises three CpG islands ((1) total range: NC_{—}000017.10 (43,224,651..43,225,293), total length: 643 bp (SEQ ID NO:6); (2) total range: NC_{—}000017.10 (43,225,464..43,226,261), total length: 798 bp (SEQ ID NO:7); and (3) total range: NC_{—}000017.10 (43,226,535..43,227,741), total length: 1,207 bp (SEQ ID NO:8)).

Data presented herein indicates that Hexim-1 plays a role in regulating obesity and diabetes, and can be used as the basis for compositions, methods and kits for treating and regulating obesity and diabetes by administering an effective amount of a Hexim-1 inhibitor, and as a basis for methods for screening for inhibitors of Hexim-1, and for screening and/or monitoring of diabetes.

### SUMMARY OF THE INVENTION

The present invention provides a method for treating and/or regulating obesity in a subject in need thereof, comprising, providing a composition comprising a Hexim-1 inhibitor or a pharmaceutical equivalent, analog, derivative or a salt thereof, and administering a therapeutically effective amount of the composition to the subject to treat obesity, thereby treating obesity in the subject.

The invention further provides a method for regulating diabetes in a subject in need thereof, comprising providing a composition comprising a Hexim-1 inhibitor or a pharmaceutical equivalent, analog, derivative or a salt thereof, and administering a therapeutically effective amount of the composition to the subject to regulate diabetes, thereby regulating diabetes in the subject. In one embodiment, diabetes is type I diabetes. In another embodiment, diabetes is type II diabetes.

Also provided herein is a method for promoting obesity and/or diabetes prophylaxis in subject in need thereof. The method comprises providing a composition comprising a Hexim-1 inhibitor or a pharmaceutical equivalent, analog, derivative or a salt thereof, and administering a therapeutically effective amount of the composition to the subject to promote obesity and/or diabetes prophylaxis, thereby promoting obesity and/or diabetes prophylaxis in the subject.

Further provided herein are methods for identifying Hexim-1 inhibitors comprising contacting Hexim-1 in Hexim-1 positive cells with a molecule of interest, and determining whether the contact results in altered expression of Hexim-1. A decrease in Hexim-1 expression, alteration in Hexim-1 function and/or signaling and/or downregulation in Hexim-1 is indicative that the molecule of interest is an inhibitor of Hexim-1.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the amino acid homology between Hexim-1 and Jak2.

FIG. 2 depicts the association between Hexim-1 and Jak2 (A) and the phosphorylation of Hexim-1 by the Jak2 kinase (B).

FIG. 3 depicts the effects of Hexim-1 on obesity in mice.

FIG. 4 depicts the effects of Hexim-1 on adipocyte cell size.

FIG. 5 depicts the effects of Hexim-1 on diabetes in mice.

FIG. 6A, upper gel panel, shows decreased expression of Hexim1 protein in primary human smooth muscle cells. The primary human smooth muscle cells were incubated with increased concentrations of an exemplary silencer 5′-UUC CAU GAA GUC GUC AUC G-3′ (SEQ ID NO:13; “miR3”) that targets Hexim1 mRNA. FIG. 6A, lower gel panel, represents the loading control showing GAPDH expression.

FIG. 6 B shows the human Hexim1 mRNA coding sequence (SEQ ID NO:10) and the relative locations within SEQ ID NO:10 of exemplary miRNA silencer sequences 5′-UUC CAU GAA GUC GUC AUC Gct-3′ (SEQ ID NO:9), 5′-CUG UAC AGU UGC UAG UUU GAG GCU G-3′ (SEQ ID NO:11; “miR1”), 5′-AUG AGG AAC UGC GUG GUG UUA-3′ (SEQ ID NO:12; “miR2”) and 5′-UUC CAU GAA GUC GUC AUC G-3′ (SEQ ID NO:13; “miR3”).

### DESCRIPTION OF EXEMPLARY ASPECTS OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Conditions” and “disease conditions,” as used herein may include, but are in no way limited to any form of diabetes and/or obesity.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

### Exemplary Therapeutic Methods

The present invention provides a method for treating obesity in a subject in need thereof, comprising, providing a composition comprising a Hexim-1 inhibitor or a pharmaceutical equivalent, analog, derivative or a salt thereof, and administering a therapeutically effective amount of the composition to the subject to treat obesity, thereby treating obesity in the subject.

The invention also provides a method for reducing obesity in a subject in need thereof, comprising providing a composition comprising a Hexim-1 inhibitor or a pharmaceutical equivalent, analog, derivative or a salt thereof, and administering a therapeutically effective amount of the composition to the subject to reduce obesity, thereby reducing obesity in the subject.

The invention further provides a method for regulating diabetes in a subject in need thereof, comprising providing a composition comprising a Hexim-1 inhibitor or a pharmaceutical equivalent, analog, derivative or a salt thereof, and administering a therapeutically effective amount of the composition to the subject to regulate diabetes, thereby regulating diabetes in the subject. In one embodiment, diabetes is type I diabetes. In another embodiment, diabetes is type II diabetes.

Also provided herein is a method for promoting obesity and/or diabetes prophylaxis in subject in need thereof. The method comprises providing a composition comprising a Hexim-1 inhibitor or a pharmaceutical equivalent, analog, derivative or a salt thereof, and administering a therapeutically effective amount of the composition to the subject to promote obesity and/or diabetes prophylaxis, thereby promoting obesity and/or diabetes prophylaxis in the subject.

The invention also provides a method for promoting weight loss in a subject in need thereof comprising providing a composition comprising a Hexim-1 inhibitor or a pharmaceutical equivalent, analog, derivative or a salt thereof, and administering a therapeutically effective amount of the composition to the subject to promote weight loss, thereby promoting weight loss in the subject.

In an embodiment of the invention, the Hexim-1 inhibitor alters (for example inhibits, reduces and/or prevents) the role of Hexim-1. Alteration of the role of Hexim-1 may be due to changes at the level of transcription and/or translation of Hexim-1 resulting in reduced expression of Hexim-1 or no expression of Hexim-1. The Hexim-1 inhibitor may also alter (for example inhibit, reduce and/or prevent) the post-translational modification (such as phosphorylation) of Hexim-1 so as to inhibit Hexim-1 or render Hexim-1 less active. Additionally, the Hexim-1 inhibitor may compete for binding the substrate/target of Hexim-1, thereby rendering Hexim-1 inactive or less active.

In some embodiments of the invention, the Hexim-1 inhibitor which inhibits and/or reduces the levels and/or activity of Hexim-1 is any one or more of a small molecule, a peptide, an antibody or a fragment thereof or a nucleic acid molecule.

In an embodiment, the nucleic acid molecule that inhibits Hexim-1 or reduces the levels and/or activity of Hexim-1 is a siRNA molecule of Hexim-1.

### Inhibition of HEXIM-1 Expression:

SiRNA.

The siRNA molecules according to the present invention mediate RNA interference (“RNAi”). The term “RNAi” is well-known in the art and is commonly understood to mean that the RNAis can be used for inhibiting gene expression, including, for example, inhibition of one or more target genes in a cell by siRNA with a region which is complementary to the target gene. RNAi can occur by way of small interfering RNA (also called short interfering RNA or silencing) (siRNA), or microRNA (miRNA). (See, for example, U.S. Pat. No. 6,506,559; Milhavet et al., Pharm. Rev. 55:629-648, 2003; and Gitlin et al., J. Virol. 77:7159-7165, 2003; incorporated herein by reference). Various assays are known in the art to test siRNA for its ability to mediate RNAi (e.g., Elbashir et al., Methods 26:199-213, 2002). The effect of the siRNA according to the present invention on gene expression will typically result in expression of the target gene being inhibited by at least 10%, 33%, 50%, 90%, 95% or 99% when compared to a cell not treated with the RNA molecules according to the present invention.

“siRNA” or “small-interfering ribonucleic acid” according to the invention has the meanings known in the art, including the following aspects. The siRNA comprises two strands of ribonucleotides which hybridize along a complementary region under physiological conditions. The strands are separate but may optionally be joined by a molecular linker. Individual ribonucleotides may be unmodified naturally occurring ribonucleotides, unmodified naturally occurring deoxyribonucleotides or may be chemically modified or synthetic (see, e.g., WO/2007/128477, incorporated herein by reference).

In particular aspects, the siRNA molecules comprise a double-stranded region which is substantially identical to a region of the mRNA of the target gene. A region with 100% identity (“fully complementary”) to the corresponding sequence of the target gene is preferred. However, the region may also contain one, two or three mismatches as compared to the corresponding region of the target gene, depending on the length of the region of the mRNA that is targeted, and as such is not necessarily fully complementary. In certain aspects, the siRNA molecules specifically target one given gene (only the desired target mRNA), and may have 100% homology to the target mRNA and, for example, at least 2 mismatched nucleotides to all other genes present in the cell or organism. Methods to identify and analyze siRNAs with sufficient sequence identity in order to effectively inhibit expression of a specific target sequence are known in the art; for example, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Another factor affecting the efficiency of the RNAi reagent is the target region of the target gene. As appreciated in the art, a suitable mRNA target region would be the coding region (e.g., in the present HEXIM-1 case nucleotide positions 1875 through 2954 of SEQ ID NO:1). Also suitable are untranslated regions, such as the 5′-UTR (e.g., in the present HEXIM-1 case nucleotide positions 1 through 1874 of SEQ ID NO:1), the 3′-UTR (e.g., in the present HEXIM-1 case nucleotide positions 2955 through 4785 of SEQ ID NO:1), and splice junctions (but no introns in HEXIM-1). Regions overlapping or adjacent the translational start site (in this HEXIM-1 case at nucleotide positions 1875-1877 of SEQ ID NO:1) may be targeted. The region of a target gene effective for inhibition by the RNAi reagent may be determined by routine experimentation. For instance, transfection assays as described in Elbashir S. M. et al., 2001 EMBO J., 20, 6877-6888 may be performed for this purpose. A number of other suitable assays and methods exist in the art which are well-known to the skilled person. Specific exemplary nucleotide sequences (e.g., oligonucleotide sequences) useful for purposes of down-regulating Hexim-1 expression are given in Example 6 below and are intended to encompass both ribonucleotide and deoxribonucleic variants of the nucleotide sequences (e.g., oligonucleotide sequences), as well as modifications as described herein.

In particular exemplary aspects, the length of the region of the siRNA complementary to the target, in accordance with the present invention, may be from 10 to 100 nucleotides, 12 to 25 nucleotides, 14 to 22 nucleotides or 15, 16, 17, 18, 19, 20 or 21 nucleotides. Where there are mismatches to the corresponding target region, the length of the complementary region will generally be somewhat longer than for fully complementary targets. Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “contacting a cell,” and any derivations thereof as used herein, refers to methods of exposing a cell, delivering to a cell, or ‘loading’ a cell with an agent (e.g., siRNA agents, antisense agents, ribozyme agents, antibodies, etc.) whether directly or indirectly by viral or non-viral vectors, and wherein such agent is bioactive upon delivery. The method of delivery will be chosen for the particular agent and use (e.g., as described herein). Parameters that affect delivery, as is known in the medical art, can include, inter alia, the cell type affected, and cellular location.

Inhibition of gene expression refers to the absence (or observable decrease) in the level of protein and/or mRNA product from a gene target. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS), inhibition of ghrelin-mediated conditions as described herein. For RNA-mediated inhibition in a cell line or whole organism, gene expression is conveniently assayed by use of a reporter or drug resistance gene whose protein product is easily assayed. Many such reporter genes are known in the art.

The invention, in particular aspects, contemplates introduction of RNA with partial or fully double-stranded character into the cell or into the extracellular environment. According to the present invention, inhibition is specific to the particular cellular gene expression product (e.g., Hexim-1) in that a nucleotide sequence from a portion of the sequence is chosen to produce inhibitory RNA. This process is effective in producing inhibition (partial or complete), and is gene-specific. In particular embodiments, the target cell containing the siRNA or miRNA may be a mammalian cell, in vitro or in vivo. Methods of preparing and using siRNA or miRNA are generally disclosed in U.S. Pat. No. 6,506,559, incorporated herein by reference (see also reviews by Milhavet et al., Pharmacological Reviews 55:629-648, 2003; Gitlin et al., J. Virol. 77:7159-7165, 2003; and WO/2007/128477, incorporated herein by reference).

The siRNA may further comprise one or more strands of polymerized ribonucleotide, and may include modifications to either the phosphate-sugar backbone or the nucleoside, and may contain non-natural amino acids (e.g., amino acid analogs). The phosphodiester linkages of natural RNA may be modified to include at least one of a nitrogen or sulfur heteroatom. Modifications in RNA structure may be tailored to allow specific genetic inhibition while avoiding a general immune-based response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. Nucleic acid containing a nucleotide sequence identical to a portion of the validated gene sequence is preferred for inhibition. RNA sequences with insertions, deletions, and single point mutations relative to the target sequence may also be effective for inhibition. Sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For siRNA (RNAi or short hairpin RNA), the RNA may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected.

Preferred siRNA compositions for oral delivery are chemically modified siRNA, as described in WO/2007/128477 (incorporated herein by reference), which confers a high in vivo stability suitable for oral delivery by including at least one modified nucleotide in at least one of the strands. Thus, the siRNA according to particular aspects contains at least one modified or non-natural ribonucleotide. A description of many known chemical modifications are disclosed in WO 200370918 (incorporated by reference herein for such teachings on known chemical modifications). Suitable modifications include, but are not limited to modifications to the sugar moiety (i.e., the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e., a non-natural or modified base which maintains ability to pair with another specific base in an alternate nucleotide chain). Other modifications include so-called ‘backbone’ modifications including, but not limited to, replacing the phosphoester group (connecting adjacent ribonuclotides with for instance phosphorothioates, chiral phosphorothioates or phosphorodithioates). Finally, end modifications sometimes referred to herein as 3′ caps or 5′ caps may be of significance. As illustrated in Table 1 of WO/2007/128477, caps may consist of simply adding additional nucleotides, such as “T-T” which has been found to confer stability on a siRNA. Caps may consist of more complex chemistries which are known to those skilled in the art.

Delivery Methods for siRNA:

In certain embodiments, siRNAs are delivered to multi-organs using amphoteric, fully charge-reversible liposomes (Reinsch C. et al., Chapter 4: Drug & Gene Delivery Systems in Nanotechnology Vol. 2, p. 328-331, incorporated by reference herein for its disclosure and teachings relating to amphoteric, fully charge-reversible liposomes).

In further embodiments, siRNAs are introduced into target cells by cyclodextrin-containing polymers, as described in U.S. Pat. Nos. 7,270,808; 7,166,302; 7,091,192; 7,018,609; 6,884,789; and 6,509,323 (incorporated by reference herein for its disclosure and teachings relating to cyclodextrin-containing polymers). These polymers form the foundation for a two-part siRNA delivery system. The first component is a linear, cyclodextrin-containing polycation that, when mixed with small interfering RNA (siRNA), binds to the anionic “backbone” of the siRNA. The polymer and siRNA self-assemble into nanoparticles of approximately 50-80 nm diameter that fully protect the siRNA from nuclease degradation in serum. The siRNA delivery system has been designed to allow for intravenous injection. Upon delivery to the target cell, the targeting ligand binds to membrane receptors on the cell surface and the RNA-containing nanoparticle is taken into the cell by endocytosis. There, chemistry built into the polymer functions to unpackage the siRNA from the delivery vehicle. Both tumors and liver cells have been effectively targeted in vivo.

In certain embodiments, siRNA is delivered to target cells using hollow yeast cell wall particles (YCWP) (Aouadi, M., et al., Chapter 4: Drug & Gene Delivery Systems in Nanotechnology Vol. 2, p. 332-335, incorporated by reference herein for its disclosure and teachings relating to hollow yeast cell wall particles (YCWP)). The siRNAs are encapsulated within these hollow YCWP by in situ layer by layer synthesis of siRNA containing nanoplexes. YCWP provide for receptor-mediated oral bioavailability and macrophage targeting of nanoplexed cargos, such as siRNA. In further embodiments, these YCWP siRNA complexes are delivered to target cells via oral and intraperitoneal route.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

RNA containing a nucleotide sequences identical to a portion of a particular gene sequence are preferred for inhibition. RNA sequences with insertions, deletions, and single point mutations relative to the target sequence may be effective for inhibition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of particular gene sequence is preferred. Alternatively, the duplex region of the RNA may be defined functionally as a nucleotide sequence that is capable of hybridizing with a portion of the particular gene transcript (e.g., 400 mM NaCl, 40 mM PIPES pH 6.4, 1 mM EDTA, 50° C. or 70° C. hybridization for 12-16 hours; followed by washing). Preferably, wherein the siRNA agent comprises a nucleic acid sequence of, e.g., at least 9, at least 15, at least 18, or at least 20 contiguous bases in length that is complementary to, or hybridizes under moderately stringent or stringent conditions to a sequence selected from the group consisting of SEQ ID NO:1, and sequences complementary thereto.

A 100% sequence identity between the RNA and a particular gene sequence is not required to practice the present invention. Thus the methods have the advantage of being able to tolerate sequence variations that might be expected due to genetic mutation, strain polymorphism, or evolutionary divergence. Sequences with greater than about 90% identity, greater than about 91% identity, greater than about 92% identity, greater than about 93% identity, greater than about 94% identity, greater than about 95% identity, greater than about 96% identity, greater than about 97% identity, greater than about 98% identity, greater than about 99% identity, greater than about 99.5% identity, greater than about 99.9% identity, or any value there between may also be used with the present invention.

Particular gene sequence siRNA may be synthesized by art-recognized methods either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands) Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (e.g., WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

The siRNA may be used alone or as a component of a composition or kit having at least one of the reagents necessary to carry out the in vitro or in vivo introduction of RNA to test samples or subjects. Preferred components are the dsRNA and a vehicle that promotes introduction of the dsRNA. Such compositions and/or kits may also include instructions to allow a user of the composition or kit to practice the invention.

Further, one or more siRNA of the present invention may comprise a pharmaceutical or therapeutic composition that may be useful, for example, as an anti-obesity agent or an anti-diabetes agent. Such pharmaceutical or therapeutic compositions may further comprise inert ingredients, such as excipients, diluents, carriers, etc.

Several methods are available in the art to produce a silencing RNA molecule, i.e. an RNA molecule which when expressed reduces the expression of a particular gene or group of genes, including the so-called “sense” or “antisense” RNA technologies.

Antisense Technology.

Thus in one embodiment, the inhibitory RNA molecule encoding chimeric gene is based on the so-called antisense technology. In other words, the coding region of the chimeric gene comprises a nucleotide sequence of at least 19 or 20 consecutive nucleotides of the complement of the nucleotide sequence of the HEXIM-1 gene or an orthologue thereof. Such a chimeric gene may be constructed by operably linking a DNA fragment comprising at least 19 or 20 nucleotides from Hexim-1 encoding gene or an orthologue thereof, isolated or identified as described elsewhere in this application, in inverse orientation to an expressible promoter and 3′ end formation region involved in transcription termination and polyadenylation.

Co-Suppression Technology.

In another embodiment, the inhibitory RNA molecule encoding chimeric gene is based on the so-called co-suppression technology. In other words, the coding region of the chimeric gene comprises a nucleotide sequence of at least 19 or 20 consecutive nucleotides of the nucleotide sequence of the HEXIM-1 gene or an ortholog thereof. Such a chimeric gene may be constructed by operably linking a DNA fragment comprising at least 19 or 20 nucleotides from the Hexim-1 encoding gene or an ortholog thereof, in direct orientation to an expressible promoter and 3′ end formation region involved in transcription termination and polyadenylation.

The efficiency of the above mentioned chimeric genes in reducing the expression of the Hexim-1 encoding gene or an ortholog thereof may be further enhanced by the inclusion of DNA element which results in the expression of aberrant, unpolyadenylated inhibitory RNA molecules or results in the retention of the inhibitory RNA molecules in the nucleus of the cells. One such DNA element suitable for that purpose is a DNA region encoding a self-splicing ribozyme, as described in WO 00/01133 (incorporated herein by reference in its entirety and particularly for its teachings on self-splicing ribozymes). Another such DNA element suitable for that purpose is a DNA region encoding an RNA nuclear localization or retention signal, as described in PCT/AUO3/00292 published as WO03/076619 (incorporated by reference).

Double-Stranded RNA (dsRNA) or Interfering RNA (RNAi).

A convenient and very efficient way of downregulating the expression of a gene of interest uses so-called double-stranded RNA (dsRNA) or interfering RNA (RNAi), as described e.g., in WO99/53050 (incorporated herein by reference in its entirety and particularly for its teachings on RNAi). In this technology, an RNA molecule is introduced into a cell, whereby the RNA molecule is capable of forming a double stranded RNA region over at least about 19 to about 21 nucleotides, and whereby one of the strands of this double stranded RNA region is about identical in nucleotide sequence to the target gene (“sense region”), whereas the other strand is about identical in nucleotide sequence to the complement of the target gene or of the sense region (“antisense region”). It is expected that for silencing of the target gene expression, the nucleotide sequence of the 19 consecutive nucleotide sequences may have one mismatch, or the sense and antisense region may differ in one nucleotide.

The length of the RNA region (sense or antisense region) may vary from about 19 nucleotides (nt) up to a length equaling the length (in nucleotides) of the endogenous gene. The total length of the sense or antisense nucleotide sequence may thus be at least at least 25 nt, or at least about 50 nt, or at least about 100 nt, or at least about 150 nt, or at least about 200 nt, or at least about 500 nt. It is expected that there is no upper limit to the total length of the sense or the antisense nucleotide sequence. However for practical reasons (such as e.g. stability of the chimeric genes) it is expected that the length of the sense or antisense nucleotide sequence should not exceed 5000 nt, particularly should not exceed 2500 nt and could be limited to about 1000 nt or about 500 nt.

It will be appreciated that the longer the total length of the sense or antisense region, the less stringent the requirements for sequence identity between these regions and the corresponding sequence in the HEXIM-1 gene and orthologs or their complements. Preferably, the nucleic acid of interest should have a sequence identity of at least about 75% with the corresponding target sequence, particularly at least about 80%, more particularly at least about 85%, quite particularly about 90%, especially about 95%, more especially about 100%, quite especially be identical to the corresponding part of the target sequence or its complement. However, it is preferred that the nucleic acid of interest always includes a sequence of about 19 consecutive nucleotides, particularly about 25 nt, more particularly about 50 nt, especially about 100 nt, quite especially about 150 nt with 100% sequence identity to the corresponding part of the target nucleic acid. Preferably, for calculating the sequence identity and designing the corresponding sense or antisense sequence, the number of gaps should be minimized, particularly for the shorter sense sequences.

dsRNA encoding chimeric genes according to the invention may comprise an intron, such as a heterologous intron, located e.g. in the spacer sequence between the sense and antisense RNA regions in accordance with the disclosure of WO 99/53050 (incorporated herein by reference).

Synthetic Micro-RNAs (miRNAs).

The use of synthetic micro-RNAs to down-regulate expression of a particular gene in a cell, provides for very high sequence specificity of the target gene, and thus allows conveniently to discriminate between closely related alleles as target genes the expression of which is to be down-regulated.

Thus, in another embodiment of the invention, the biologically active RNA or silencing RNA or inhibitory RNA molecule may be a microRNA molecule, designed, synthesized and/or modulated to target and cause the Hexim-1 encoding gene or an ortholog thereof. Various methods have been described to generate and use miRNAs for a specific target. Usually, an existing miRNA scaffold is modified in the target gene recognizing portion so that the generated miRNA now guides the RISC complex to cleave the RNA molecules transcribed from the target nucleic acid. miRNA scaffolds could be modified or synthesized such that the miRNA now comprises 21 consecutive nucleotides of the Hexim-1 encoding nucleotide sequence or an ortholog thereof, such as the sequences represented in the Sequence Listing, and allowing mismatches according to the herein below described rules.

A miRNA is processed from a “pre-miRNA” molecule by proteins, such as Dicer proteins, and loaded onto a RISC complex where it can guide the cleavage of the target RNA molecules.

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop and further comprising the nucleotide sequence of the miRNA (and its complement sequence) in the double stranded RNA stem. Preferably, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

In another embodiment of the invention, an antibody which inhibits Hexim-1 and/or reduces the levels and/or activity of Hexim-1, is any one or more of a monoclonal antibody or fragment thereof, a polyclonal antibody or a fragment thereof, a chimeric antibody, a humanized antibody, a human antibody or a single chain antibody.

The subjects treated by the present invention include mammalian subjects, including, human, monkey, ape, dog, cat, cow, horse, goat, pig, rabbit, mouse and rat.

Various methods may be utilized to administer the composition comprising a Hexim-1 inhibitor, including but not limited to aerosol, nasal, oral, intravenous, intramuscular, intraperitoneal, inhalation, transmucosal, transdermal, parenteral, implantable pump, continuous infusion, topical application, capsules and/or injections.

Typical dosages of an effective amount of a Hexim-1 inhibitor can be in the ranges recommended by the manufacturer where known therapeutic compounds are used, and also as indicated to the skilled artisan by the in vitro responses or responses in animal models. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

### Exemplary Screening Methods

Another aspect of the invention relates to assays and methods for identifying compounds that inhibit Hexim-1. In one embodiment, the method comprises contacting Hexim-1 in a Hexim-1 positive cell with the compound of interest and subsequently determining whether the contact results in altered amounts of Hexim-1. In an embodiment of the claimed methods, an alteration in the amount of Hexim-1 is a decrease in the amount of Hexim-1. In one embodiment, a decrease in the amount of Hexim-1 is indicative that the molecule of interest is an inhibitor of Hexim-1. In another embodiment, decrease in the amount of Hexim-1 synthesized is indicative that the molecule of interest is an inhibitor of Hexim-1. In a further embodiment, decrease in the amount of nucleic acid (for example, mRNA) encoding Hexim-1 is indicative that the molecule of interest is an inhibitor of Hexim-1.

The compound of interest that inhibits Hexim-1 may be any one or more of a small organic/inorganic molecule, a peptide, an antibody or a fragment thereof and a nucleic acid molecule.

Assays that may be employed to identify compounds that inhibit Hexim-1 include but are not limited to microarray assay, quantitative PCR, Northern blot assay, Southern blot assay, Western blot assay immunohistochemical assays, binding assays, gel retardation assays or assays using yeast two-hybrid systems. A person skilled in the art can readily employ numerous techniques known in the art to determine whether a particular agent inhibits Hexim-1.

Exemplary Pharmaceutical Compositions

In various embodiments, the present invention provides pharmaceutical compositions including a pharmaceutically acceptable excipient along with a therapeutically effective amount of a Hexim-1 inhibitor. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

### Exemplary Kits

The present invention is also directed to kits to treat and/or regulate obesity and diabetes, etc., as described herein. The kit is an assemblage of materials or components, including at least one of the inventive compositions. Thus, in some embodiments the kit contains a composition including a Hexim-1 inhibitor, as described herein.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat and/or regulate obesity and/or diabetes in a subject. Optionally, the kit also contains other useful components, such as, measuring tools, diluents, buffers, pharmaceutically acceptable carrier(s), containers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing a Hexim-1 inhibitor. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Hexim-1 was Determined to Share the Conserved YXXL Motif Present in Jak2 Kinase

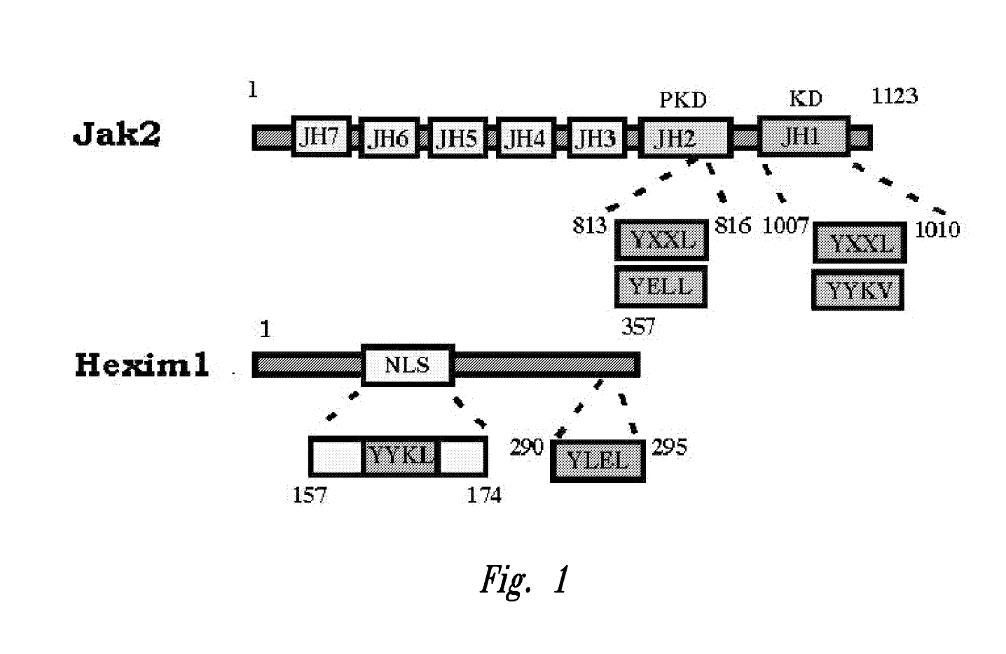

Amino-acid homology analysis revealed that Hexim-1 shares the conserved YXXL motif present in Jak2 kinase and the adaptor SH2B1 (FIG. 1).

To establish whether Jak2 kinase phosphorylates the conserved YXXL present in Hexim-1, a constitutively activate Jak2 kinase (TEL-Jak2) was co-transfected with Hexim-1 expression vector (pcCLP1).

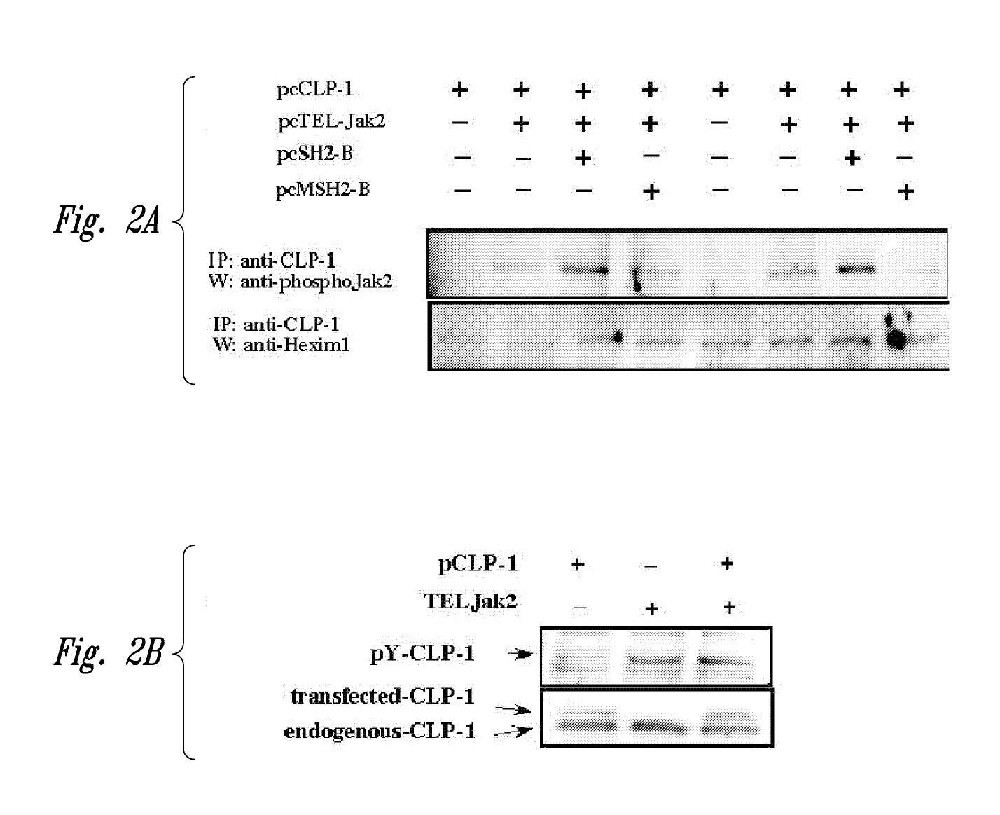

FIG. 2A shows the association between the phosphorylated Jak2 kinase and Hexim1.

FIG. 2B shows the phosphorylation of Hexim-1 by Jak2 kinase.

According to particular aspects, therefore, Hexim-1 shares the conserved YXXL motif present in Jak2 kinase, and is phosphorylated by Jak2 kinase.

### Example 2

### Downregulation of Hexim-1 Expression Resulted in Reduced Increase of Body Weight, and Precluded Leptin Resistance

Knock-out mutations of the mouse homologue of Hexim-1, cardiac lineage protein (CLP-I), conferred embryonic lethality. However, the heterozygous mice are viable but highly susceptible to stress.

In order to evaluate the role of Hexim-1 in obesity and/or diabetes wild type mice and Hexim-1 heterozygous mice were subjected to High Fat Diet (HFD) for eight weeks. The metabolic changes and cardiovascular parameters in both groups were subsequently evaluated.

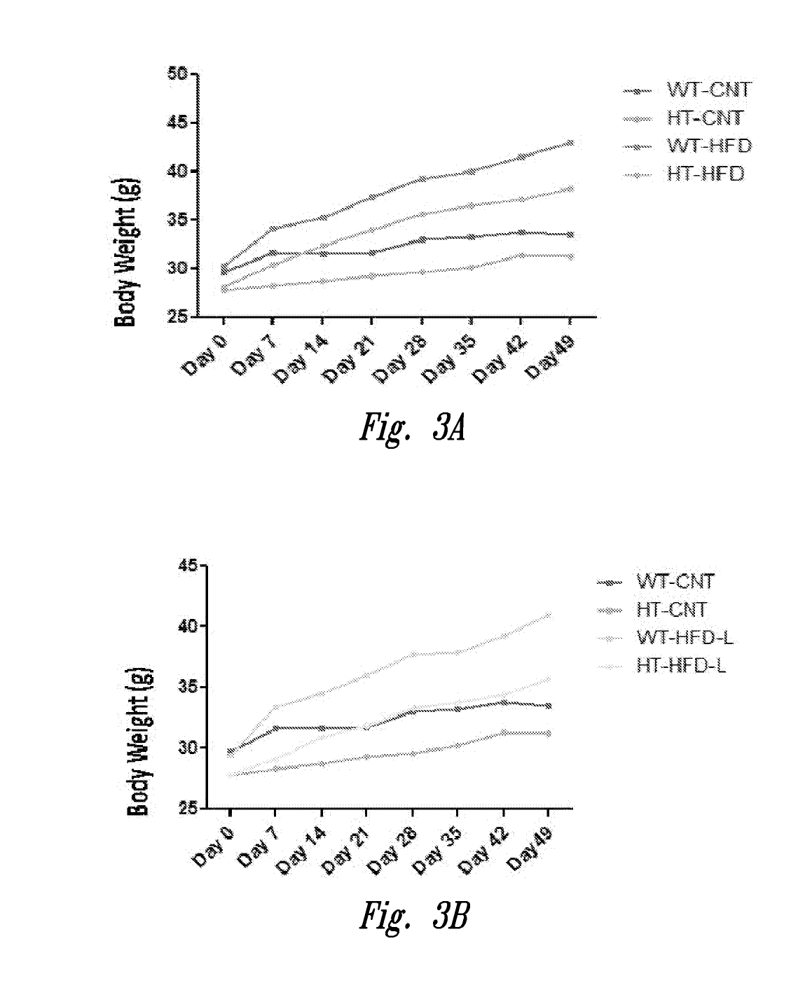

FIG. 3A shows that the increase in body weight is significantly less in Hexim-1 heterozygous mice, and FIG. 3B shows that Hexim-1 heterozygous mice subjected to HFD do not develop leptin resistance and consequently are more susceptible to maintain a low body weight due to leptin treatment.

According to particular aspects, downregulation of Hexim-1 expression provides a method for treating obesity (e.g., weight gain) and/or preventing of reducing leptin resistance.

### Example 3

### Downregulation of Hexim-1 Expression Precluded Hypertrophy of Adipocytes in Mice Subjected to HFD

At the cellular level obesity is characterized by enlargement of the fat cells or adipocytes (hypertrophy).

The adipocyte cell size in each mice group of Example 2 (heterozygous and WT) was evaluated.

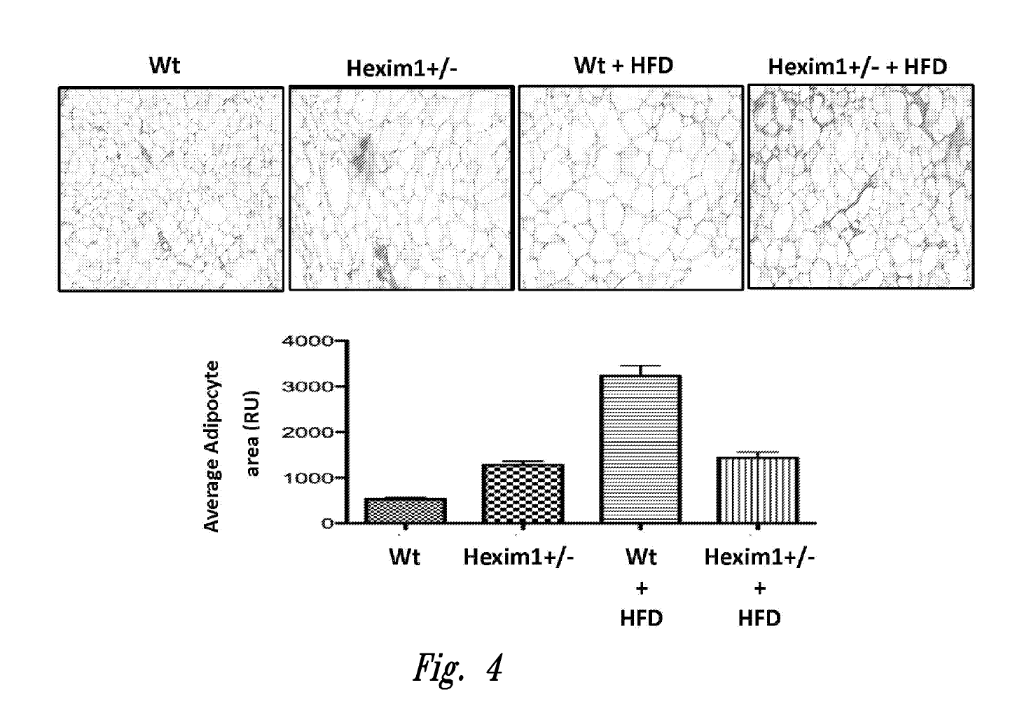

As seen in FIG. 4, HFD triggered hypertrophy of adipocytes in the wild type mice. However, the adipocytes in Hexim-1 heterozygous mice remained unchanged. These observations are consistent with the absence of obesity observed in the Hexim-1 heterozygous mice subjected to HFD (FIG. 4).

According to particular aspects, downregulation of Hexim-1 expression provides a method for precluding or reducing hypertrophy of adipocytes.

### Example 4

### Downregulation of Hexim-1 Expression Precluded or Reduced Hyperglycemia in Mice Subjected to HFD

A significant feature of the HFD model in mice is not only the increase in body weight but also the development of insulin resistance of diabetes type II.

To evaluate the glycemic state of each group of mice of Example 2 (heterozygous and WT), a glucose tolerance test was performed.

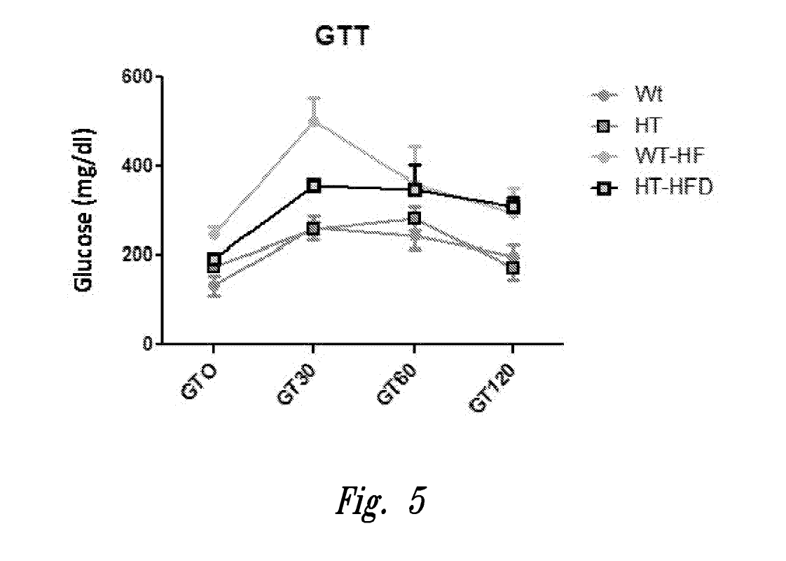

As shown in FIG. 5, only wild type mice revealed high levels of glucose (diabetes). Hexim-1 heterozygous mice showed an enhanced glucose clearance system when subjected to HFD.

According to particular aspects, therefore, the data indicate that modulation (e.g., reduction) of the level of expression of Hexim-1 and/or activity has substantial utility as a method for treating obesity and diabetes, and/or for preventing of reducing leptin resistance, and/or for precluding or reducing hypertrophy of adipocytes.

### Example 5

### Hexim-1 Heterozygous Mice Failed to Display Induced SOCS3 Expression

According to additional aspects, prior to the current disclosure, there was no evidence of a role for Hexim-1 in regulating metabolic activity as described herein. Additional evidence also showed that compared to wildtype, Hexim-1 heterozygous mice did not show induced SOCS3 expression.

As discussed above, elevated SOCS3 expression is a consistent finding in obesity and diabetes.

Data presented herein provides important evidence that Hexim-1 shares key structural motif with already established regulators of leptin signaling, including Jak2 kinase and the IRS-I. Indeed, amino-acid homology analysis revealed that Hexim-1 shares the conserved YXXL and is tyrosine phosphorylated by Jak2 kinase. As shown in FIG. 1, there are at least two YXXL motifs in the Hexim-1 protein. The in vivo data described herein clearly established that Hexim-1 plays a role in regulating obesity and diabetes.

### Example 6

### Exemplarly Oligonucleotide Sequences for Use in Down-Regulation of Hexim-1 Expression

According to additional aspects, and with respect to down-regulation of Hexim-1 expression as discussed above (e.g., siRNA reagents, etc., as described above), examples of inventive nucleic acid (e.g., oligonucleotide sequences) of length X (in nucleotides), as indicated by polynucleotide positions with reference to, for example, SEQ ID NO:1, include those corresponding to sets (sets corresponding to both the sense and antisense sequences of SEQ ID NO:1) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));

where n=1, 2, 3, . . . (Y−(X−1));

where Y equals the length (nucleotides or base pairs) of SEQ ID NO:1 (4785);

where X equals the common length (in nucleotides) of each oligonucleotide in the set (e.g., X=19 for a set of consecutively overlapping 19-mers); and

where the number (Z) of consecutively overlapping oligomers of length X for a given SEQ ID NO of length Y is equal to Y−(X−1). For example Z=4785−18=4767 for either sense or antisense sets of SEQ ID NO:1, where X=19.

Examples of inventive 19-mer oligonucleotides include the following set of 4767 oligomers (and the antisense set complementary thereto), indicated by polynucleotide positions with reference to SEQ ID NO:1:

1-19, 2-20, 3-21, 4-22, 5-23, . . . and 4767-4785.

Likewise, examples of inventive 25-mer oligonucleotides include the following set of 4761 oligomers (and the antisense set complementary thereto), indicated by polynucleotide positions with reference to SEQ ID NO:1:

1-25, 2-26, 3-27, 4-28, 5-29, . . . and 4761-4785.

The present invention, for example, encompasses, for SEQ ID NO:1 (sets corresponding to both the sense and antisense sequences of SEQ ID NO:1), multiple consecutively overlapping sets of oligonucleotides or modified oligonucleotides of length X, where, for example, X=9, 10, 11, 12, 13, 14, 15, 16, 17, 18, 19, 20, 21, 22, 23, 24, 25, 26, 27, 28, 29, 30 or greater (e.g., 35 nucleotides in length).

The oligonucleotides or oligomers or RNAi agents (e.g, siRNA agents, etc.), according to the present invention provide for practicing the disclosed methods. Preferred sets of such oligonucleotides or modified oligonucleotides of length X are those consecutively overlapping sets of oligomers corresponding to SEQ ID NO:1 and to the complements thereof. Likewise for SEQ ID NO:3 (mouse).

### Example 7

### Targeted Downregulation of Hexim1 Protein was Demonstrated Using an Exemplary siHexim1-Specific Antisense Approach

This example shows targeted downregulation of Hexim1 protein by an exemplary siHexim1-specific antisense approach.

Primary human smooth muscle cells were incubated with increased concentrations of the silencer UUCCAUGAAGUCGUCAUCG (SEQ ID NO:13; “miR3”) that targets the Hexim1 mRNA within its coding sequence SEQ ID NO:10 (the target corresponding to nucleotides 2585-2603 of SEQ ID NO:1), where SEQ ID NO:13 is complementary to nucleotides 2585-2603 of SEQ ID NO:1.

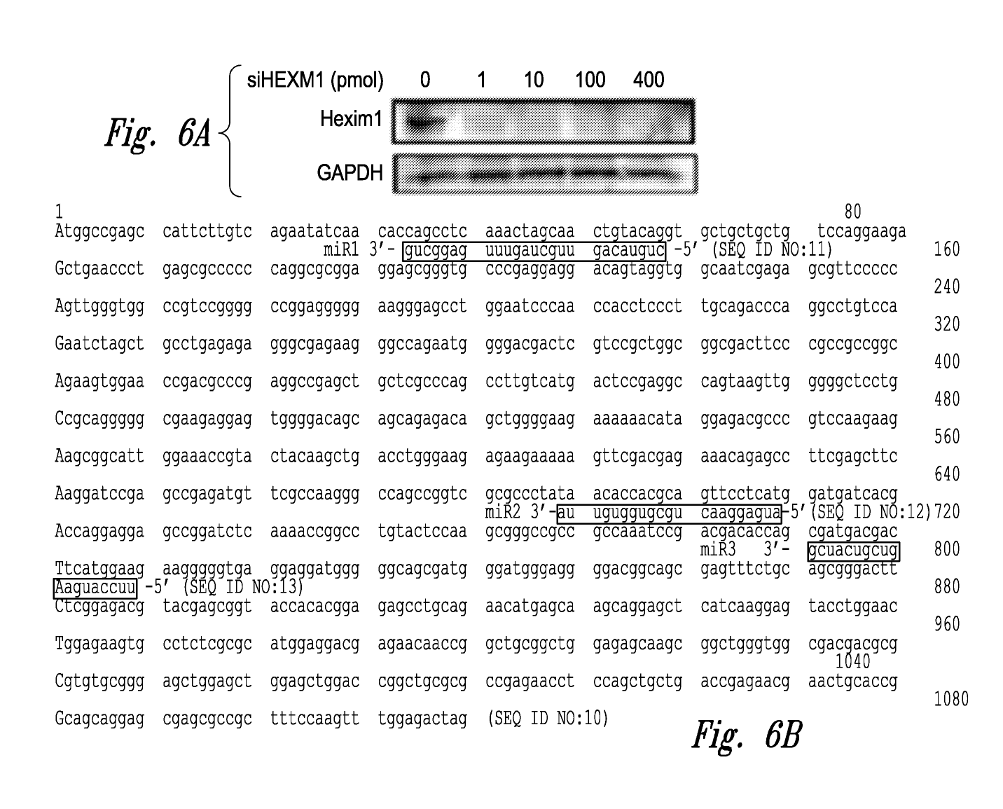

FIG. 6A, upper gel panel, shows decreased expression of Hexim1 protein in primary human smooth muscle cells. FIG. 6A, lower gel panel, represents the loading control showing GAPDH expression.

FIG. 6 B shows the human Hexim1 mRNA coding sequence (SEQ ID NO:10) and the target location of the miRNA silencer sequence (SEQ ID NO:13; “miR3”).

In addition to the silencer 5′-UUC CAU GAA GUC GUC AUC G-3′ (SEQ ID NO:13′ miR3), exemplary miRNA silencer sequence 5′-UUC CAU GAA GUC GUC AUC Gct-3′ (SEQ ID NO:9) was likewise shown to be effective in targeting Hexim-1 mRNA and decreasing the synthesis of Hexim-1 protein in primary human smooth muscle cells.

In further embodiments, the above described exemplary miRNA silencer sequences, and additional exemplary miRNA silencer sequences 5′-CUG UAC AGU UGC UAG UUU GAG GCU G-3′ (SEQ ID NO:11; “miR1”) and 5′-AUG AGG AAC UGC GUG GUG UUA-3′ (SEQ ID NO:12; “miR2”) were likewise shown to be effective in targeting Hexim-1 mRNA and decreasing the synthesis of Hexim-1 protein in the human cell lines 293T, LNCaP, PC3 and HepG2 cells.

### REFERENCES, INCORPORATED BY REFERENCE HEREIN

1. Martin S S, Qasim A, Reilly M P. Leptin resistance: a possible interface of inflammation and metabolism in obesity-related cardiovascular disease. J Am Coll Cardiol. Oct. 7, 2008; 52(15):1201-1210.
2. Myers M G, Cowley M A, Munzberg H. Mechanisms of leptin action and leptin resistance. Annu Rev Physiol. 2008; 70:537-556.
3. Velloso L A, Folli F, Sun X J, et al. Cross-talk between the insulin and angiotensin signaling systems. Proc Natl Acad Sci USA. Oct. 29, 1996; 93(22):12490-12495.
4. Howard J K, Flier J S. Attenuation of leptin and insulin signaling by SOCS proteins. Trends Endocrinol Metab. November 2006; 17(9):365-371.
5. Maures T J, Kurzer J H, Carter-Su C. SH2B1 (SH2-B) and JAK2: a multifunctional adaptor protein and kinase made for each other. Trends Endocrinol Metab. January-February 2007; 18(1):38-45.
6. Zhou Q, Yik J H. The Yin and Yang of P-TEFb regulation: implications for human immunodeficiency virus gene expression and global control of cell growth and differentiation. Microbiol Mol Biol Rev. September 2006; 70(3):646-659.
7. Dey A, Chao S H, Lane D P. HEXIM1 and the control of transcription elongation: from cancer and inflammation to AIDS and cardiac hypertrophy. Cell Cycle. Aug. 1, 2007; 6(15):1856-1863.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicants at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

We claim:

1. A method for treating or reducing obesity or for treating or reducing diabetes, comprising administering, to a subject in need thereof, a therapeutically effective amount of a Hexim-1 inhibitor.

2. (canceled)

3. (canceled)

4. (canceled)

5. The method of claim 1, wherein the Hexim-1 inhibitor is selected from the group consisting of a nucleic acid molecule, small molecule, a peptide, and an antibody or an epitope-binding portion or fragment thereof.

6. The method of claim 1, wherein weight loss is promoted in the subject.

7. The method of claim 5, wherein the nucleic acid molecule comprises an siRNA or an miRNA molecule of Hexim-1 or an siRNA or an miRNA molecule which is specific to Hexim-1.

8. The method of claim 5, wherein the antibody is selected from the group consisting of
a monoclonal antibody or an epitope-binding portion or fragment thereof,
a polyclonal antibody or an epitope-binding portion or fragment thereof,
a chimeric antibody,
a humanized antibody,
a human antibody, and
a single chain antibody.

9. The method of claim 1, wherein the Hexim-1 inhibitor is administered intravenously, intramuscularly, intraperitoneally, orally or via inhalation.

10. A method of claim 1, wherein the effective amount of the Hexim-1 inhibitor is about 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day or 1900-2000 mg/day.

11. A method for identifying inhibitors of Hexim-1 comprising:
contacting Hexim-1 in Hexim-1 positive cells with a molecule of interest, and
determining whether the contact results in decreased expression and/or activity of Hexim-1, wherein a decrease in Hexim-1 expression and/or activity is indicative that the molecule of interest is an inhibitor of Hexim-1.

12. The method of claim 11, wherein the Hexim-1 inhibitor is selected from the group consisting of a nucleic acid molecule, a small molecule, a peptide, and an antibody or an epitope-binding portion or a fragment thereof.

13. The method of claim 11, wherein contacting comprises separately contacting each of a plurality of samples or molecules of interest to be tested.

14. The method of claim 13, wherein the plurality of samples or molecules of interest comprises more than about 104 samples or molecules of interest.

15. The method of claim 13, wherein the plurality of samples or molecules of interest comprises more than about 5×10⁴ samples or molecules of interest.

16. The method of claim 1, wherein the subject is selected from the group consisting of human, non-human primate, monkey, ape, dog, cat, cow, horse, rabbit, mouse and rat.

17. A kit or a pharmaceutical composition for the treatment of obesity, regulating obesity, or treating or regulating diabetes, comprising:
a composition comprising a Hexim-1 inhibitor; and
instructions for use of the composition for the treatment of obesity, regulating obesity, or treating or regulating diabetes.

18. The kit of claim 17, wherein the Hexim-1 inhibitor comprises a nucleic acid molecule comprising an siRNA or an miRNA molecule of Hexim-1 or an siRNA or an miRNA molecule which is specific to Hexim-1.

19. The kit of claim 18, wherein the Hexim-1 inhibitor is an siRNA or an miRNA having greater than 90% sequence identity to Hexim-1 comprising the cDNA sequence of SEQ ID NO: 10.

20. The kit of claim 18, wherein the inhibitor is a silencer miRNA having the sequence set forth in (a) 5′-UUC CAU GAA GUC GUC AUC Gct-3′ (SEQ ID NO:9); (b) 5′-CUG UAC AGU UGC UAG UUU GAG GCU G-3′ (SEQ ID NO: 11); (c) 5′-AUG AGG AAC UGC GUG GUG UUA-3′ (SEQ ID NO: 12); or (d) 5′-UUC CAU GAA GUC GUC AUC G-3′ (SEQ ID NO: 13).
